(19) United States
(12) Patent Application Publication
(10) Pub. No.: US 2010/0130787 A1
(43) Pub. Date: May 27, 2010

(54) REACTION PLATES WITH ALTERNATIVE, UNORDERED MICROSTRUCTURED SURFACES FOR MICROREACTORS FOR PERFORMING GAS-LIQUID REACTIONS

(76) Inventors: Sascha Braune, Luftenberg am Donau (AT); Stefan Steinhofer, Gurten (AT)

Correspondence Address: NIXON & VANDERHYE, PC, 901 NORTH GLEBE ROAD, 11TH FLOOR, ARLINGTON, VA, 22203, US

(21) Appl. No.: 12/532,220

(22) PCT Filed: Mar. 18, 2008
(86) PCT No.: PCT/EP08/53232

§ 371 Date: Jan. 14, 2010

(30) Foreign Application Priority Data
Mar. 22, 2007 (AT) A 449/2007

(51) Int. Cl. B01J 19/00; B01J 10/02; B01J 7/02; C07C 45/33; C07C 209/00

(52) U.S. Cl. 564/413; 422/129; 422/240; 422/239; 568/431

(57) ABSTRACT

Reaction plates for microreactors for performing gas-liquid reactions, which consist of falling-film plates with a surface which has a randomly distributed, unordered fine structure or microstructure.

The present invention relates to reaction plates with alternative, unordered microstructured surfaces for microreactors and to their use for gas-liquid reactions.

To avoid safety problems in strongly exothermic reactions, it is proposed in the literature to perform these reactions in a microreactor. In micro-reactors, these reactions are easier to control than in conventional batch reactors. In addition, it is possible in the microreactor to realize reaction conditions which are not realizable for safety reasons in a classical method in the laboratory or on the industrial scale.

Reactions which can lead to safety problems are, for example, gas-liquid reactions, for instance catalytic hydrogenations, oxidations, for instance ozonolysis; halogenation with gaseous chlorine or fluorine, alkoxylations with gaseous epoxides, addition of hydrogen halides onto double bonds, phosgenations, acidic esterifications, for example with isobutene; or reactions with ammonia.

Microreactors for gas-liquid reactions are, for example, those with continuous phase flow, for instance a micro-falling-film absorber, and are supplied by various manufacturers.

Micro-falling-film absorbers are based on the principle of the wetting of surfaces by a liquid film under the influence of gravity. An important constituent of the reactor, according to the prior art, is a so-called reaction sheet or reaction plate with microstructure. According to the prior art, this microstructure is present in ordered form and has, for example, the form of microchannels which, depending on the material of the reaction sheet, the desired dimensions and geometric ratios, can be obtained by different preparation processes.

Known preparation processes are, for example, anisotropic wet etching of microcrystalline materials, the dry etching process, the LIGA process, etc. The commercially available ordered microstructures are, however, obtained only with a high level of machine complexity and, associated with this, high production costs. In the case of micro-falling-film absorbers, for this purpose, a multitude of microchannels is incorporated into the reaction plate in parallel, such that a strictly ordered structure is obtained. In addition, time- and labor-intensive production of the microstructures is necessary in order to achieve laminar flow in the falling film. A micro-falling-film absorber as used to date in the prior art is described, for example, in Ind. Eng. Chem. Res., Vol. 44, No. 25, 2005, page 9751.

It was an object of the present invention to find reaction plates which are inexpensive and simple to produce and, at the same time, ensure at least equal or increased throughput, selectivity and yields in gas-liquid reactions compared to the prior art.

Unexpectedly, this object was achieved by a metal plate which has a randomly distributed, unordered fine structure or microstructure.

The present invention therefore provides reaction plates for microreactors for performing gas-liquid reactions, which consist of falling-film plates with a surface which has a randomly distributed, unordered fine structure or microstructure.

In the simplest case, the inventive reaction plates consist of a metal plate which can be structured by very simple machining methods, for example by sanding by hand with sandpaper, but alternatively also by material-removing machining techniques, for example by milling, engraving, dry or wet grinding, brushing, sandblasting, grit blasting or the action of glass beads. However, the structuring can also be effected by laser engraving, etching or pickling.

In the gas-liquid reaction, this plate is arranged in the microreactor such that the liquid flows along the surface of this plate only owing to the influence of gravity and, as it does so, comes into contact with the gaseous reactant and reacts.

Unexpectedly, when this simply machined reaction plate is used, at least equal or even increased yields and selectivities are obtained in gas-liquid reactions compared to those with commercially available microreactors which comprise reaction plates with microstructures produced in a complicated manner.

Suitable materials for the falling-film plate are metals, for instance steels such as 1.4571, Hastelloy and the like, or metals in general which behave inertly toward the reaction media. However, glass, silicon carbide or chemically inert plastics are also suitable for use as a falling-film plate. The most important property is chemical inertness toward the reaction media, sufficient stiffness under pressure, sufficient thermal conductivity to ensure temperature control and sufficiently easy machinability.

Preference is given to using metals inert toward the reaction media, more preferably steels, for instance 1.4571.

The size of the falling-film plate is adjusted to the size of the reactor housing analogously to the commercially available falling-film microreactors. In the case of a laboratory microreactor, plates with a gas-liquid contact zone of a width of from about 0.5 to 50 cm and a length of from about 1 to 100 cm can be used; the plates preferably have a gas-liquid contact zone of a width of from 2.0 cm to 6 cm and a length of from 3.0 cm to 50.0 cm. Particular preference is given to plates having a gas-liquid contact zone of a width of from 2.0 to 3.0 cm and a length of from 6.0 to 10.0 cm, and to those having a gas-liquid contact zone with a width of from 4.0 to 6.0 cm and a length of from 25.0 to 35.0 cm.

The shape of the plates is likewise adjusted to the shape of the reactor housing. According to the microreactor used, flat plates, but also twisted, curved or rolled reaction plates on which a falling film can be formed, are useful.

In contrast to the ordered microstructured plates according to the prior art, the slots for the liquid inlet and outlet in the inventive plates can be configured flexibly.

For example, the slots, with a width of 2.5 cm, may have a height between 0.2 and 2.5 mm. At a width of 5.3 cm, the height of the slots may, for example, be 2.0 mm.

The thickness of the plate has no relevant influence on the chemical reactions. The plate must only be sufficiently pressure-stable that there are no leaks owing to deformations or fracture. A further criterion for the thickness is a sufficient thermal conductivity to be able to ensure sufficient temperature control during the reaction. Moreover, the plate must be fitted exactly into the reactor housing.

The randomly distributed, unordered microstructure may, as mentioned above, be applied to the smooth plate in a simple manner. For example, this can be done by simple sanding or roughening of the plate, for example with coarse sandpaper by hand. Alternatively, the structures can be introduced into a smooth plate by material-removing machining techniques, for example by milling, engraving, dry or wet grinding, brushing, sandblasting, grit blasting or the action of glass beads. However, the structuring can also be effected by laser engraving, etching or pickling. Preference is given to effecting the structuring by simple sanding or roughening, brushing or by laser engraving.

Particular preference is given to simple sanding or roughening.

The structures may have orders of magnitude which lie starting in the nanostructured region up into the millimeter- or centimeter-structured region. In the case of the sanded metal plate, for example, roughness with a grain size of 40 or 80 are preferred, which in this case means an engraving depth of about 0.5 μm or about 20 μm. It is additionally found to be advantageous when the structure is structured in liquid direction. A homogeneous falling film thus forms more easily, which does not tend to inhomogenization at the end of the falling-film plate.

A further possibility is to cover the smooth plate with a network like structure. Networklike structures are, for example, nylon fabric, as, for instance, in women's stockings, or other meshlike network or fabric of different materials, for example Teflon fabric, steel wool, glass wool or other natural or synthetic polymers which are inert and stable toward the reaction media. The mesh width of the networks is within a range from 0.01 to 5 mm, but preferably within the order of magnitude from 0.2 to 0.7 mm. The thickness of the fabric must be adjusted to the dimensions of the reactor housing and thus lie within a range of from 0.01 to 5 mm.

These measures afford surface structures which increase the residence time of the liquid in the microreactor and the surface area required for the phase transition or ensure the formation of a homogeneous falling film with simple means. As a result, at least the throughput, the selectivity and the yields in the gas-liquid reactions according to the prior art are likewise achieved, and in some cases even increased.

The inventive reaction plates are suitable for incorporation into microreactors for gas-liquid reactions. Suitable microreactors are known and are supplied commercially, for example, by the Institut für Mikrotechnik, Mainz GmbH (IMM), Cellular Process Chemistry GmbH or Mikroglas AG. Preference is given to falling-film absorbers, as described, for instance, in Ind. Eng. Chem. Res., Vol. 44, No. 25, 2005, page 9751. In principle, though, the alternatively structured, unordered fine structures can be used in all reactor types which are based on the formation of a falling film as the basic principle.

Gas-liquid reactions are catalytic or noncatalytic hydrogenations, oxidations, for instance ozonolysis; halogenation with gaseous chlorine or fluorine, alkoxylations with gaseous epoxides, addition of hydrogen halides onto double bonds, phosgenations, acidic esterifications, for example with isobutene; or reactions with ammonia.

Preference is given to using the inventive reaction plates in oxidations, particular preference to using them in ozonolysis processes.

A further important advantage of the alternatively structured, unordered microstructures consists in the possibility of being able to vary the falling-film thicknesses. In the case of the conventional microstructures, the liquid phase is forced through the slot into the channels. The fact that the liquid flows only into the channels is ensured by virtue of a frame resting on the elements of the structure and thus the liquid only being able to flow in through the channels. In the case of the alternatively structured, unordered fine structure, the resting frame would close the slots, and no liquid could get into the reaction chamber.

Consequently, in the inventive plates, a so-called spacer in the form of a frame which leaves the slots of the inventive plate open is inserted between falling-film plate and boundary of the gas space as a spacer. The thickness of this spacer can be used to adjust the thickness of the falling film. The spacer may consist of any material resistant to reaction media, such as the corresponding steels 1.4571, Hastelloy or of Teflon, etc. The distance between boundary and falling-film plate, which is defined by the thickness of the spacer, may be within a range between 0.001 mm and 5 mm, preferably from 0.01 to 2.0 mm.

The spacer may, though, also be incorporated or integrated directly into the falling-film plate or into the gas space boundary.

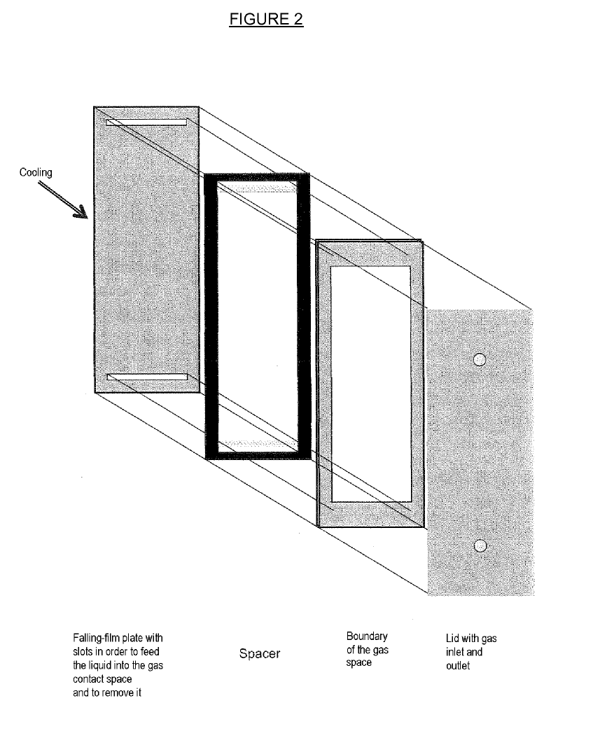

An illustrative arrangement of a falling-film plate, spacer and boundary of the gas space is evident from FIG. 2.

### EXAMPLES

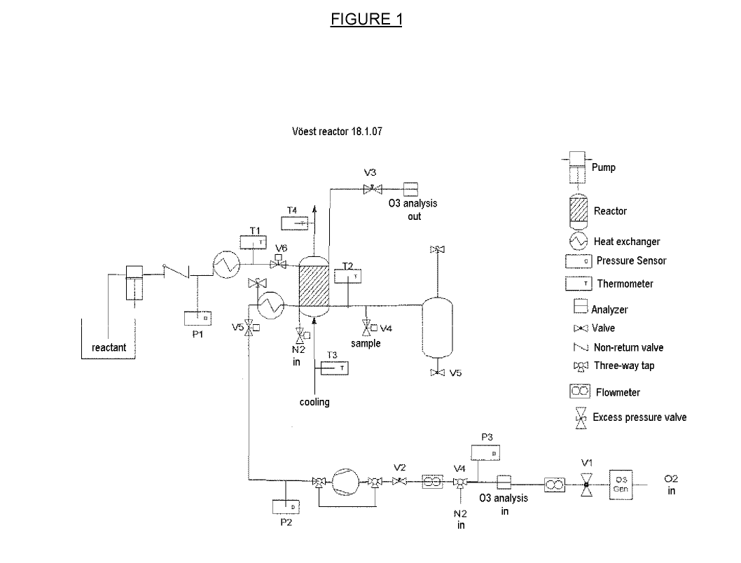

Various ozonolyses were carried out in an apparatus according to FIG. 1:

The microreactor used was a falling-film absorber from the Institut für Mikrotechnologie in Mainz with a falling-film plate which consists of 32 channels with a channel depth and width of 0.6 mm, as depicted, for instance, in Ind. Eng. Chem. Res., Vol. 44, No. 25, 2005, page 9751, the commercial reaction plate having been replaced in each case by an inventive reaction plate. The falling-film plates sanded by hand had a width of 2.5 cm and a length of 8 mm in the region of the gas-liquid contacting. The slots were 2.5 cm wide and 0.2 mm or 2.5 mm high. The spacer used was a Teflon frame with a thickness of 0.5 mm.

In each case, comparative tests were carried out with the original reaction plate (original microstructure, 32 channels, in each case with a channel depth and width of 600×600 μm).

### Example 1

### Preparation of O-Phthalaldehyde (OPA) by Ozonolysis of Naphthalene

The reaction plate used was a metal plate (1.4571 steel, width of the gas-liquid contact area 2.5 cm and length of the gas-liquid contact area 8.0 cm) roughened in longitudinal direction with coarse sandpaper (grain size 40).

### Reaction Parameters:

| | |
|---|---|
| Liquid phase: | 2% naphthalene in methanol |
| Gas pressure absolute: | 5 bar |
| Liquid phase flow: | Ismatek pump 2 g/min |
| Gas flow: | 1 l (STP)/min |
| Gas composition: | ozone/oxygen mixture |
| Ozone concentration in: | 130 g/m³ (STP) |
| Cryotemp. for reactor: | −30° C. |
| Cryotemp. for preliminary cooling: | −30° C. |

At the start of the experiment, the naphthalene solution was pumped into the reactor under nitrogen atmosphere with a flow rate of 2 g/min. The product vessel was initially charged with a 30% w/w bis(2-ethanol) sulfide solution in ethyl acetate. After switching to oxygen, the gas was compressed to 5 bar, and the ozone generator was regulated to the desired ozone content. After 10 minutes, all reaction parameters showed constant values:

p(O₂ᵢₙ)=2 bar; p₁=5.2 bar; p₂=5.1 bar; p₃=1.1 bar;
m(O₃ᵢₙ)/V(O₂)=130 g O₃/m³(STP)O₂; m(O₃ₒᵤₜ)/V(O₂)=85 g O₃/m³(STP)O₂;
v(gas flow)=1 l(STP)/min;

### T₁=−19° C.; T₂=−3° C.; T₃=−20° C.; T₄=−19° C.

In order to obtain analytical samples, small amounts of the reaction mixture were reduced directly to the product with a solution of 30% w/w of bis 2-ethanol) sulfide in ethyl acetate, and quantified by gas chromatography.

### Result:

| Conversion of | | |
|---|---|---|
| naphthalene [%] | Yield of OPA synthons [%] | Selectivity [%] |
| 71.7 | 48.5 | 67.6 |

Comparative Experiment with Original Plate:

| Conversion of | | |
|---|---|---|
| naphthalene [%] | Yield of OPA synthons [%] | Selectivity [%] |
| 59.7 | 37.8 | 63.3 |

### Example 2

Analogously to Example 1, naphthalene was converted to OPA by ozonolysis.

The reaction plate used was a smooth metal plate with a nylon fabric stretched over it (width of the gas-liquid contact area 2.5 cm and length of the gas-liquid contact area 8.0 cm, mesh width 0.5 mm).

### Result:

| Conversion of | | |
|---|---|---|
| naphthalene [%] | Yield of OPA [%] | Selectivity [%] |
| 71.2 | 42.0 | 59.0 |

### Example 3

### Preparation of N-acetylaminoacetophenone (N-AAAP) from 2,3-dimethyl-indole

### Reaction Parameters:

| | |
|---|---|
| Liquid phase: | 2% wt of 2,3-dimethylindole in |
| | methanol |
| Ozone pressure absolute: | 1 bar |
| Liquid phase flow: | Ismatek pump 2 g/min |
| Ozone flow: | 1 l (STP)/min |
| Ozone concentration in: | 32 g/m³ (STP) |
| Cryotemp. for reactor: | −30° C. |
| Cryotemp. for preliminary cooling: | −30° C. |

Procedure was analogous to Example 1.

| | Conversion of | |
|---|---|---|
| Reaction plate | dimethylindole [%] | N-AAAP yield [%] |
| Original microstructure | 100% | 89 |
| Original microstructure + | 70-88 | 57-75 |
| nylon fabric | | |
| Smooth plate with | | |
| nylon fabric | 100% | 94% |
| Roughened plate | 100% | 93% |

1. A reaction plate for microreactors for performing gas-liquid reactions, which consists of falling-film plates with a surface which has a randomly distributed, unordered fine structure or microstructure.

2. The reaction plate as claimed in claim 1, wherein the plate consists of a metal inert toward the reaction media, of glass, silicon carbide or a plastic inert toward the reaction media.

3. The reaction plate as claimed in claim 1, wherein the randomly distributed, unordered fine structure or microstructure is applied to the plate by sanding or roughening, milling, engraving, dry or wet grinding, brushing, sandblasting, grit or glass bead blasting, laser engraving, etching or pickling.

4. The reaction plate as claimed in claim 1, wherein the randomly distributed, unordered fine structure or microstructure is applied by covering the plate with a nylon fabric or a meshlike network or fabric which is composed of materials inert toward the reaction media and is selected from the group of Teflon fabric, steel wool, glass wool or natural or synthetic polymer fabric.

5. The use of reaction plates as claimed in claim 1 for incorporation into microreactors for gas-liquid reactions.

6. The use of reaction plates as claimed in claim 1 for incorporation into microreactor types which are based on the formation of a falling film as the basic principle.

7. The use of reaction plates as claimed in claim 1 in gas-liquid reactions from the group of catalytic hydrogenations, oxidations, halogenation with gaseous chlorine or fluorine, alkoxylations with gaseous epoxides, addition of hydrogen halides onto double bonds, phosgenations, acidic esterifications, or reactions with ammonia.

8. The use of reaction plates as claimed in claim 1 in combination with a spacer which is mounted between falling-film plate and boundary of the gas space in the form of a frame which leaves the slots of the reaction plate open.
